(19) United States
(12) Patent Application Publication
(10) Pub. No.: US 2012/0277580 A1
(43) Pub. Date: Nov. 1, 2012

(54) SYSTEM AND METHOD FOR DILUTING BLOOD IN A VESSEL

(75) Inventors: Anthony Van LE, San Jose, CA (US); Nicholas John RICHARDI, Manteca, CA (US)

(73) Assignee: POINCARE SYSTEMS, INC., Menlo Park, CA (US)

(21) Appl. No.: 13/098,097

(22) Filed: Apr. 29, 2011

(51) Int. Cl. A61B 6/00

(52) U.S. Cl. 600/427

(57) ABSTRACT

A device for delivering fluid inside a vessel includes an elongated member having a proximal end, a distal end, and a channel extending between the proximal end and the distal end, and a first port located at the distal end, wherein the first port is in fluid communication with the channel, wherein the first port faces at least partially towards a proximal direction.

### FIELD

This application relates to systems and methods for delivering fluid inside a body, and more particularly, to systems and methods for delivering fluid to dilute blood in a vessel.

### BACKGROUND

Imaging devices have been used inside a body to obtain images of tissue inside the body. Examples of such imaging devices include fluoroscopic device, CMOS device, ultrasound device, and laser-based imaging device (such as optical coherence tomography device). These imaging devices generally include an imaging window through which image signals are received. Sometimes, during an imaging procedure, substance may be on or near the imaging window, thereby preventing image signals from being obtained. For example, when the imaging device is inserted into a vessel, blood may interfere with the imaging procedure. Applicant of the subject application determines that it would be desirable to provide a system and a method for removing or at least reducing an amount of such substance during an imaging procedure.

### SUMMARY

In accordance with some embodiments, a device for delivering fluid inside a vessel includes an elongated member having a proximal end, a distal end, and a channel extending between the proximal end and the distal end, and a first port located at the distal end, wherein the first port is in fluid communication with the channel, wherein the first port faces at least partially towards a proximal direction.

In accordance with other embodiments, a device for delivering fluid inside a vessel includes an elongated member having a proximal end, a distal end, and a channel extending between the proximal end and the distal end, a first port located at the distal end, wherein the first port is in fluid communication with the channel; and an imaging window, wherein the first port is configured to deliver a fluid to dilute blood inside the vessel before the diluted blood reaches the imaging window.

Other and further aspects and features will be evident from reading the following detailed description of the embodiments.

### BRIEF DESCRIPTION OF THE DRAWINGS

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments and are not therefore to be considered limiting of its scope.

FIG. 1 illustrates a fluid delivery device in accordance with some embodiments, particularly showing the device delivering fluid downstream so that the fluid flows in a same direction as blood flow;

FIG. 2 illustrates the fluid delivery device of FIG. 1, particularly showing the device delivering fluid against blood flow in accordance with other embodiments;

FIG. 3 illustrates another fluid delivery device in accordance with other embodiments, particularly showing the distal end of the device without an enlarged profile;

FIG. 4 illustrates another fluid delivery device in accordance with other embodiments, particularly showing the distal end of the device without an enlarged profile;

FIG. 4A illustrates another fluid delivery device in accordance with other embodiments;

FIG. 5 illustrates another fluid delivery device in accordance with other embodiments, particularly showing the fluid delivery device being integrated with an imaging device;

FIG. 6 illustrates another fluid delivery device in accordance with other embodiments, particularly showing a channel within a wall of an elongated member;

FIG. 7 illustrates another fluid delivery device in accordance with other embodiments, particularly showing an imaging device being moveable relative to fluid delivery ports, and is placed distal to the ports;

FIG. 8 illustrates the fluid delivery device of FIG. 7, particularly showing the imaging device being placed proximal to the fluid delivery ports;

FIG. 9 illustrates another fluid delivery device in accordance with other embodiments, particularly showing the fluid delivery device having a pressure sensor;

FIG. 10 illustrates another fluid delivery device in accordance with other embodiments;

FIG. 11 illustrates another fluid delivery device in accordance with other embodiments; and

FIG. 12 illustrates another fluid delivery device in accordance with other embodiments.

### DESCRIPTION OF THE EMBODIMENTS

Various embodiments are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the claimed invention or as a limitation on the scope of the claimed invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated or described.

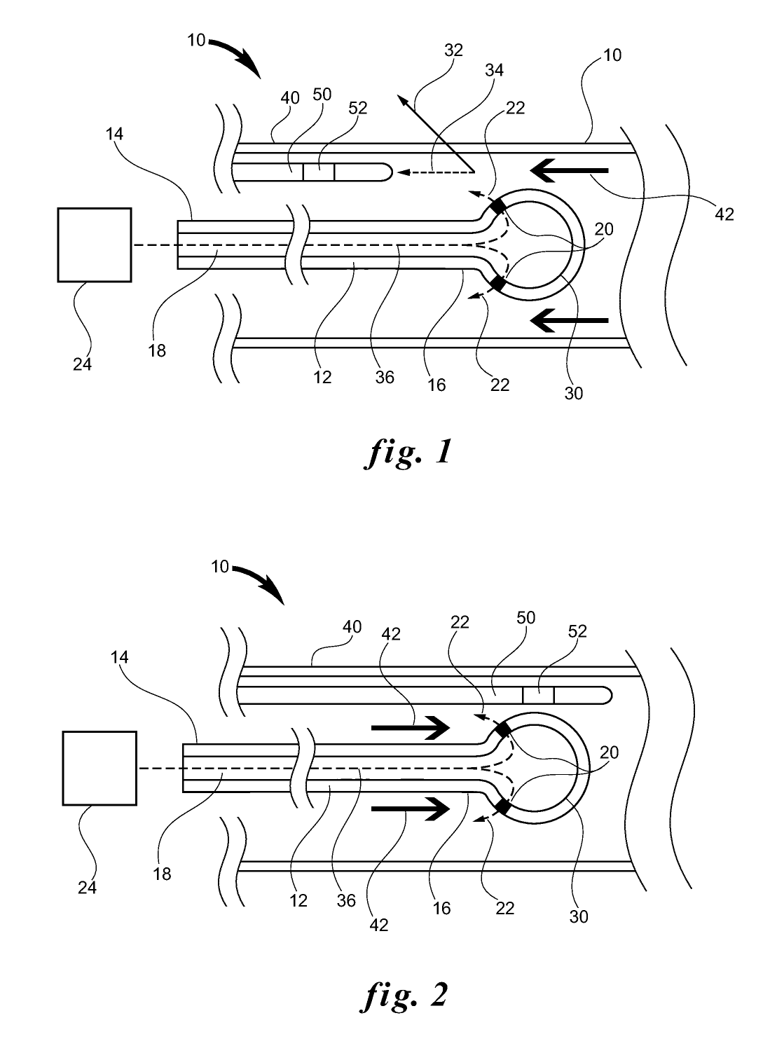

FIG. 1 illustrates a fluid delivery device 10 in accordance with some embodiments. The fluid delivery device 10 includes an elongated member 12 having a proximal end 14, a distal end 16, and a channel 18 extending between the proximal end 14 and the distal end 16. The fluid delivery device 10 also includes two ports 20 at the distal end 16. Although two ports 20 are shown, in other embodiments, the fluid delivery device 10 may include only one port 20, or more than two ports 20. If the fluid delivery device 10 includes a plurality of ports 20, the ports 20 may be disposed circumferentially at the outer surface of the elongated member 12. The ports 20 are configured to deliver fluid 22 from a fluid source 24 that is coupled to the proximal end 14 of the elongated member 12 during use. In some embodiments, the fluid source 24 may be considered as a part of the fluid delivery device 10. The fluid source 24 may include any fluid, such as saline, drug, contrast, ringers lactate solution, radio-opaque fluid (such as Visopaque™), or other agent. Also, in some embodiments, the fluid source 24 may include substitute fluid that can function as blood substitute, including but not limited to fluid that contains water, fluids hydroethyl starch (Trade name Tetrastartch), hydroethylcellulose, Polyethylene glycol (PEG), and/or Dextran.

In the illustrated embodiments, the port 20 is located at a wall 30 at the distal end 16 of the elongated member 12. The port 20 faces towards a direction 32 that is at least partially towards a proximal direction. In particular, the port 20 faces towards a direction 32 having a directional component 34 that is pointing towards a proximal direction. In other embodiments, the ports 20 may be facing completely towards the proximal direction. In such cases, the wall 30 at which the ports 20 are located may be perpendicular to the longitudinal axis 36 of the elongated member 12, so that the wall 30 is facing completely towards the proximal direction.

The fluid delivery device 10 may be used with an imaging device 50 having an imaging window 52. In any of the embodiments described herein, the imaging device 50 may be considered to be a part of an imaging system that includes both the imaging device 50 and the fluid delivery device 10. Thus, as used in this specification, the term “fluid delivery device” or similar terms may refer to an imaging device/system (or a component of it), and is not limited to a device that can only deliver fluid.

In some embodiments, the elongated member 12 and the imaging device 50 may be coupled together. For example, in some embodiments, the device 10 may further include a sheath with one or more lumens for housing at least a part of the elongated member and at least a part of the imaging device 50.

During use, the fluid delivery device 10 is inserted percutaneously into a patient, and the distal end 16 of the fluid delivery device 10 is placed inside a vessel 40 (artery or vein). The fluid delivery device 10 is then advanced inside the vessel 40 until it reaches a target location at which imaging is desired to be performed. As shown in the figure, the imaging device 50 with the imaging window 52 is placed inside the vessel 40, where blood flow 42 is moving from a distal direction towards a proximal direction. In the illustrated embodiments, the ports 20 of the fluid delivery device 10 are placed at an operative position that is distal to the imaging window 52 of the imaging device 50, and are configured to deliver the fluid 22 for diluting blood. Such technique is advantageous because it allows the fluid 22 to be mixed with the blood before the diluted blood reaches the imaging window 52 of the imaging device 50. The diluted blood allows the imaging device 50 to more effectively obtain image signals through the imaging window 52. In some cases, the blood cells in the blood that cause optical scattering may be diluted out (e.g., reduced in concentration), or may be replaced. For example, a fluid that is compatible as a blood replacement may be introduced using the embodiments of the device described herein. Such fluid may displace the blood completely, thereby resulting in a portion of the fluid inside the vessel that is completely free of particles. The introduced fluid may be visopaque™ and/or viscous (e.g., more viscous than blood).

FIG. 2 illustrates the same fluid delivery device 10 of FIG. 1, particularly showing the device 10 being used to delivery fluid against blood flow. In the illustrated embodiments, the distal end of the imaging device 50 is facing downstream of the blood flow 42. The ports 20 of the fluid delivery device 10 are placed at an operative position that is proximal to the imaging window 52 of the imaging device 50. Such technique is advantageous in that it allows the fluid 22 from the ports 20 to be mixed with the blood before the diluted blood reaches the imaging window 52 of the imaging device 50. Also delivering fluid 22 against the blood flow 42 is advantageous because it may create uneven flow (e.g., turbulence) within the blood vessel 40 to thereby allow the fluid 22 to be more effectively mixed with the blood flow 42.

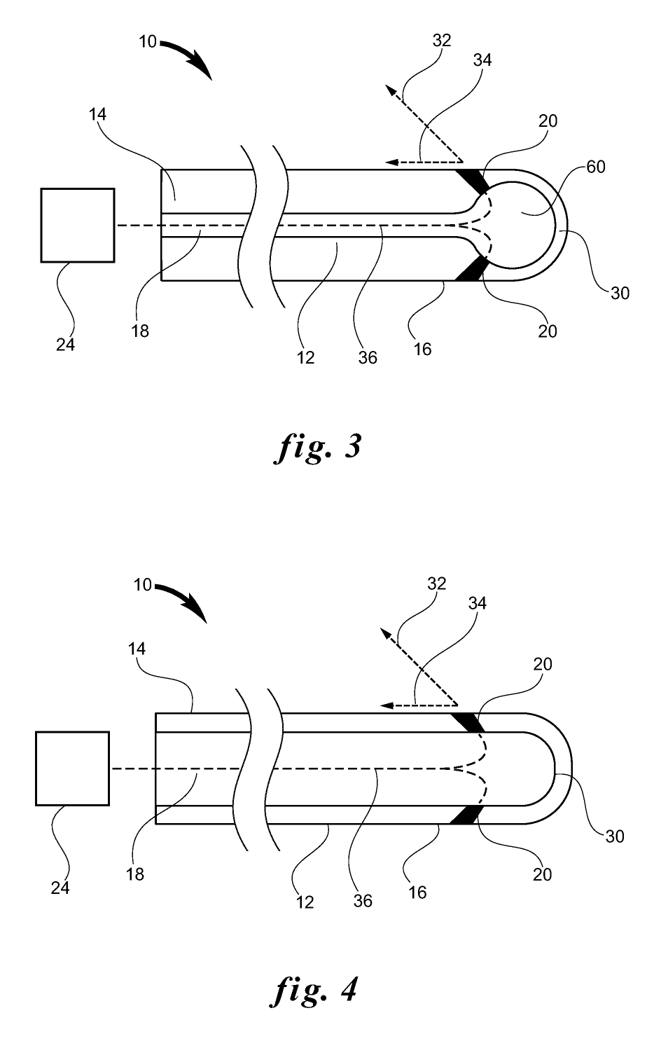

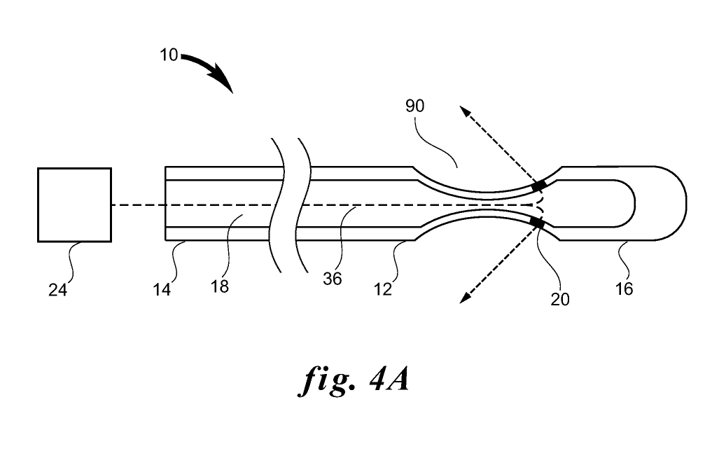

In the above embodiments, the elongated member 12 has an enlarged portion at the distal end 16 so that the wall 30 at which the ports 20 are located is facing at least partially towards a proximal direction. Such configuration allows the ports 20 to be facing at least partially towards the proximal direction. In other embodiments, the wall 30 of the elongated member 12 may have an exterior surface that is approximately parallel (e.g., ±5°) to the longitudinal axis 36 of the elongated member 12 (FIG. 3). Such configuration allows the fluid delivery device 10 to have a small profile throughout the distal portion, without any enlarged profile. In the illustrated embodiments, the distal end 16 of the elongated member 12 has a lumen 60 with a cross section that is larger than a cross sectional dimension of the channel 18. In other embodiments, the elongated member 12 may not include the enlarged lumen 60 (FIG. 4), in which cases, the channel 18 with a constant cross sectional shape extends from the proximal end 14 to the distal end 16.

In alternative embodiments, the elongated member 12 may have a narrowed configuration at an intermediate section 90 of the member 12 (FIG. 4A). Such configuration allows the elongated member 12 to have a substantially constant cross sectional shape (e.g., a constant cross sectional shape along a majority of the length of the member 12).

In any of the embodiments described herein, the imaging device 50 may be placed in the lumen 18 of the device 10. In such cases, the device 10 may include an imaging window for allowing the imaging device 50 to view outside the device 10 therethrough.

In any of the embodiments described herein, the fluid delivery device 10 may include an imaging window, and may be integrated with an imaging device. FIG. 5 illustrates a fluid delivery/imaging device 10 in accordance with some embodiments. The device 10 provides imaging capability as well as fluid delivery capability. The device 10 includes an elongated member 12 having a proximal end 14, a distal end 16, and a lumen 18 extending between the proximal end 14 and the distal end 16. The device 10 also includes two ports 20 at the distal end 16 for delivering fluid, and respective tubes 100 connecting the two ports 20 to the fluid source 24. Each of the tubes 100 includes a channel therewithin that is in fluid communication with a corresponding port 20. The tubes 100 are housed by the elongated member 12 and are located in the lumen 18 of the elongated member 12. In other embodiments, the tubes 100 may be located in a wall of the elongated member 12. As similarly discussed, in other embodiments, the device 10 may include only one port 20 or more than two ports 20 (in which cases, the ports 20 may be disposed circumferentially at a surface of the elongated member 12). Also as similarly discussed, the port 20 is facing at a direction 32 that is at least partially towards a proximal direction. Such configuration allows fluid to be delivered out of the port 20 in a direction that is at least partially towards the proximal direction.

In the illustrated embodiments, the device 10 also includes an imaging window 102 at the distal end 16. The imaging window 102 is located distal to the ports 20.

As shown in the figure, the device 10 also includes an imaging device 110 configured to perform optical coherence tomography. The imaging device 110 includes a rotor 112 housed and rotatably supported within the lumen 18 of the elongated member 12, electromagnetic coil(s) 114 for turning the rotor 112, a beam director 116 (e.g., a mirror or a prism), an optical component 118 (which may be one or a combination of a focusing lens, a collimation lens, a plano convex lens, a bi-convex lens, a gradient index lens, and a finite conjugate lens), and an optical waveguide 120. The rotor 112 includes a first channel 142 and a second channel 144 for allowing light to travel therethrough. The imaging device 110 also includes a control 124, an interferometer 126, a laser source 128, and a processing module 129. The interferometer 126 is optically coupled to the waveguide 120 through the optical connection 127 during use. The laser source 128 is configured to provide a broadband input light to the interferometer 126. In the illustrated embodiments, the input light is in an infrared range. In some embodiments, the input light has a center wavelength that is anywhere between 100 nm and 11000 nm, and more preferably, anywhere between 1000 nm and 2000 nm, and even more preferably anywhere between 1100 nm and 1600 nm (such as 1310 nm). In other embodiments, the input light may have other wavelengths. The interferometer 126 passes the input light to a fiber optic that transmits the input light to the inside of the elongated member 12. The input light is processed optically (e.g., focused, collimated, reflected, etc.) by the components 118, 112, inside the device 10, and the processed input light is output through the imaging window 102 of the device 10 as an output light. In the illustrated embodiments, the output light has a wavelength that is anywhere between 100 nm and 11000 nm, and more preferably anywhere between 500 nm and 1500 nm, and even more preferably anywhere between 12100 nm and 1400 nm (such as 1310 nm). In other embodiments, the output light may have other wavelengths. It should be noted that the term “light” or similar terms (such as “light beam”) is not limited to non-visible light, and may refer to any radiation in different wavelengths, which may or may not be visible.

The output light from the device 10 impinges onto a tissue within a patient, and is reflected from the tissue. The reflected light from the tissue is then captured by the device 10 through region 102, is optically processed inside the device 10, and is then transmitted by fiber optic back to the interferometer 126. The interferometer 126 passes the light signal from the waveguide 120 to the processing module 129. The processing module 129 detects and processes the signal, and transmits it to the user interface 138. In the illustrated embodiments, the processing module 129 includes one or more photodetector(s) 130, a signal amplifier or conditioner with an ant-alias filter 132, an A/D converter 134, and a Fast Fourier Transform (FFT) processor 136. The photodetector(s) 130 is configured to detect light containing the depth encoded interferogram from interferometer 126, and convert the light to electrical signal(s). The electrical signals are further conditioned and amplified by the component 132 to be suitable for use by the A/D converter 134. Once the signal is converted from the analog domain to digital domain by the A/D converter 134, the FFT processor 136 converts the depth encoded electrical interferogram signal via FFT to a depth resolved signal for each point scanned by the device 10. The FFT processor 136 maybe a discrete processing board, or maybe implemented by a computer. The user interface 138 may be a computer (as illustrated), a hand-held device, or any of other devices that is capable of presenting information to the user. The user interface 138 reconstructs the image from the FFT processor 136 and display a result (e.g., an image) of the processing in a screen for the user's viewing.

The delivering of output light by the device 10, and the receiving of reflected light by the device 10, may be repeated at different angles circumferentially around the device 10, thereby resulting in a circumferential scan of tissue that is located around the imaging device 10. In some embodiments, one or more components within the distal end of the device 10 are configured to rotate at several thousand times per minute, and the associated electronics for processing the light signals are very fast, e.g., has a sample rate of 180,000,000 times a second. In other embodiments, the one or more components within the distal end of the device 10 may rotate at other speeds that are different from that described previously. Also, in other embodiments, the associated electronics for processing the light signals may have a data processing speed that is different from that described previously.

The control 124 is connected to the device 10 via electrical connection 148. The electrical connection 148 may be used to control functions of the device 10, as well a providing power to magnetic coils 114 to turn the rotor 112 located distally in the device 10. In some embodiments, the control 124 may be used to control a positioning of one or more optical components located inside the device 10. For example, in some embodiments, the control 124 may have a manual control for allowing a user to control a turning (e.g., amount of turn, speed of turn, angular position, etc.) of the beam director 116 which directs the light beam 146 to exit through the region 102 at different angles.

In other embodiments, the control 124 may having a manual control for allowing a user to move one or more lens inside the device 10 so that a focusing function may be performed. In further embodiments, the control 124 may have a switch which allows a user to select between manual focusing, or auto-focusing.

When auto-focusing is selected, the imaging device 110 will perform focusing automatically. In still further embodiments, the control 124 may also includes one or more controls for allowing a user to operate the imaging device 110 during use (e.g., to start image scanning, stop image scanning, etc.).

In any of the embodiments described herein, the device 10 is flexible and is steerable using the control 124. In such cases, the device 10 may include a steering mechanism for steering the distal end 16 of the elongated member 12.

For example, the steering mechanism may include one or more wires coupled to the distal end 16 of the elongated member 12, wherein tension may be applied to any one of the wires using the control 124. In particular, the control 124 may include a manual control that mechanically couples to the wire(s). During use, the user may operate the manual control to apply tension to a selected one of the wires, thereby resulting in the distal end 16 bending in a certain direction.

Although the imaging window 100 is illustrated as located distal to the ports 20, in other embodiments, the imaging window 100 may be located proximal to the ports 20. In such cases, the imaging components housed inside the elongated member 12 may be located at a corresponding position that is proximal to the ports 20. During use, the distal tip of the device 10 will be facing against blood flow, and fluid delivered from the ports 20 will be mixed with blood travelling proximally before the diluted blood reaches the imaging window 100.

Although the above embodiments of the device 10 have been described with reference to optical coherence tomography. In other embodiments, the imaging device 110 that is incorporated in, or used with, the device 10 may be configured to perform different types of imaging, such as mulitphoton imaging, confocal imaging, Raman spectroscopy, spectroscopy, scanning imaging spectroscopy, and Raman spectroscopic imaging. In further embodiments, the imaging device 110 may be a fluoroscope, an ultrasonic imaging device, or any of other types of imaging device.

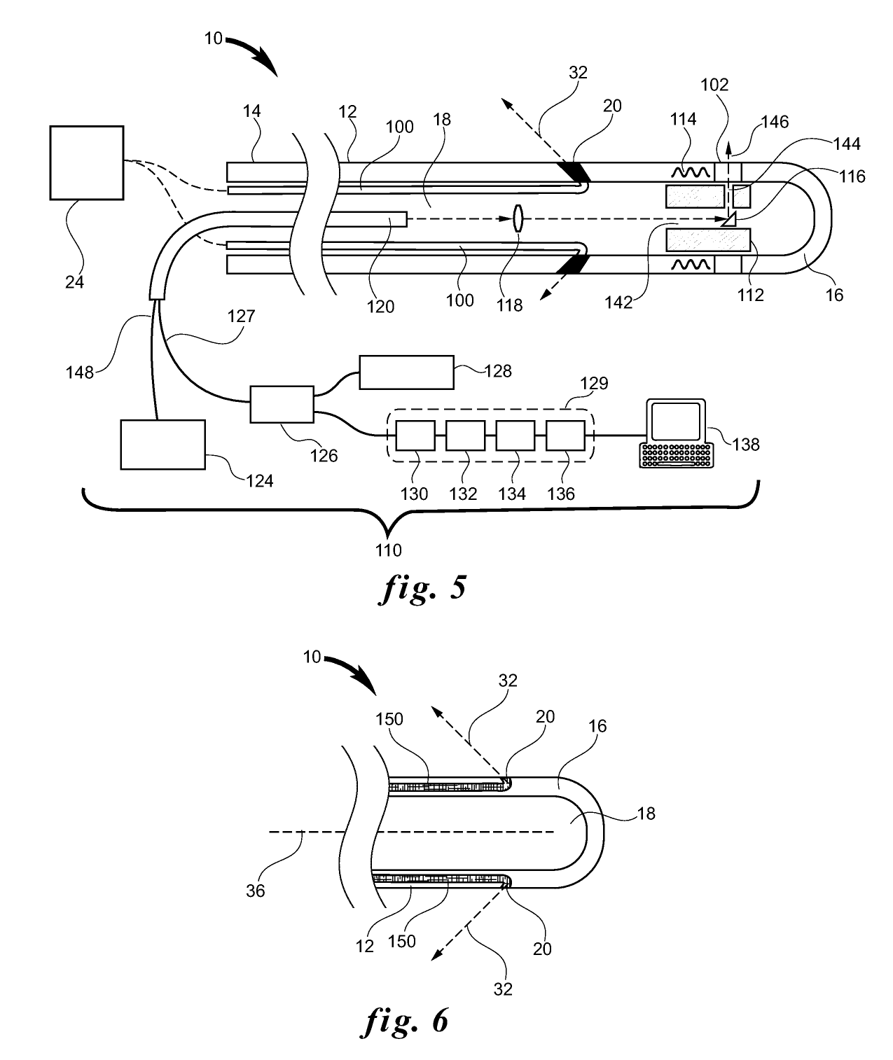

In the above embodiments, the ports 20 are illustrated as coupling to respective tubes for delivering fluid from the fluid source 24. In other embodiments, the device 10 may include only one tube for delivering fluid to one or a plurality of ports 20. Also, in other embodiments, the device 10 may not include any tube for delivering fluid from the fluid source 24 to the port(s) 20. Instead, the elongated member 12 of the device 10 may include one or more channels 150 inside the wall 152 of the elongated member 12 for delivering fluid from the fluid source 24 to the ports 20 (FIG. 6).

In any of the embodiments described herein the imaging device 50/110 may be integrated as a part of the device 10 that is translatable relative to the elongated member 12. FIG. 7 illustrates another fluid delivery device 10 in accordance with other embodiments. In the illustrated embodiments, the device 10 includes the elongated member 12 having the proximal end 14 and the distal end 16. The device 10 also includes the port(s) 20, as similarly discussed previously. The device 10 also includes one or more tubes 100 (not shown for clarity) for delivering fluid from the fluid source 24 to the port(s) 20, as similarly discussed with reference to FIG. 5. Alternatively, the device 10 may include one or more channels 150 in the wall of the elongated member 12 for delivering the fluid, as similarly discussed with reference to FIG. 6. As shown in the figure, the device 10 also includes a first imaging window 102a, and a second imaging window 102b. The imaging device 50 with its imaging window 52 is placed within the lumen 18 of the elongated member 12. Thus, the elongated member 12 and the imaging device 50 are translatably coupled to each other.

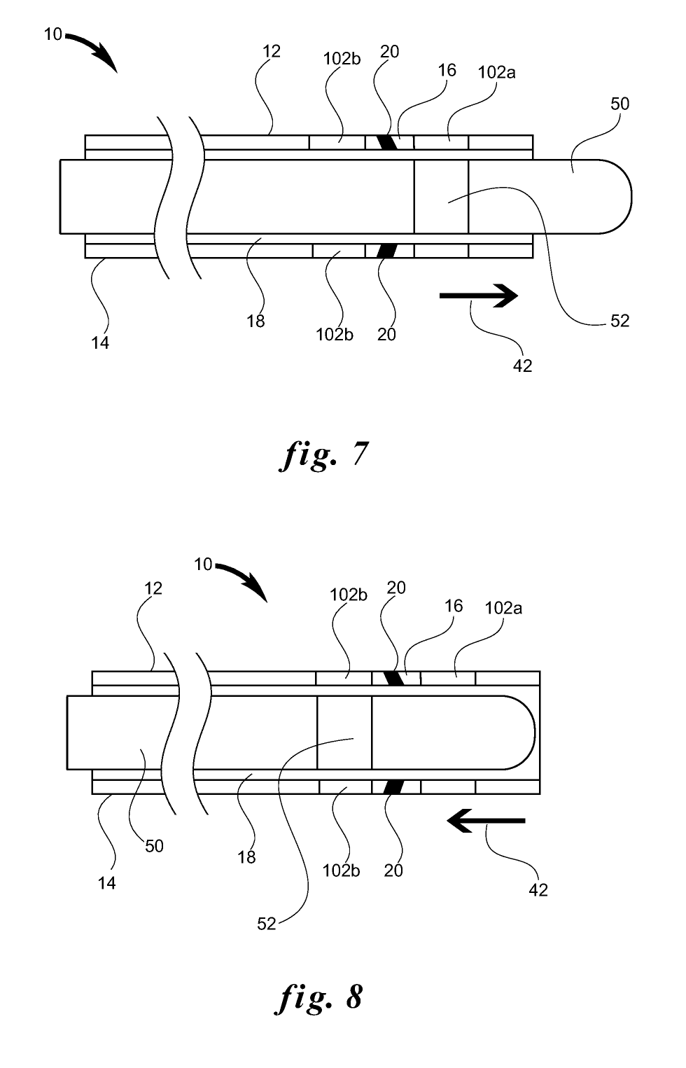

During use, the imaging device 50 may be translated axially relative to the tubular member 12, so that its imaging window 52 may be selectively placed next to the first imaging window 102a (FIG. 7), or the second imaging window 102b (FIG. 8). For example, in one method of use, blood may be flowing towards a distal direction (as indicated by the blood flow arrow 42 in FIG. 7). In such cases, the imaging device 50 is advanced distally (or the elongated member 12 may be retracted proximally) so that its imaging window 52 is aligned with the first imaging window 102a. Fluid is then delivered through the ports 20 so that it is mixed with the blood. The above configuration is advantageous because it ensures that the fluid has diluted the blood before the diluted blood reaches the imaging window 102a.

In another method of use, blood may be flowing towards a proximal direction (as indicated by the blood flow arrow 42 in FIG. 8). In such cases, the imaging device 50 is retracted proximally (or the elongated member 12 may be advanced distally) so that its imaging window 52 is aligned with the second imaging window 102a. Fluid is then delivered through the ports 20 so that it is mixed with the blood. The above configuration is advantageous because it ensures that the fluid has diluted the blood before the diluted blood reaches the imaging window 102b.

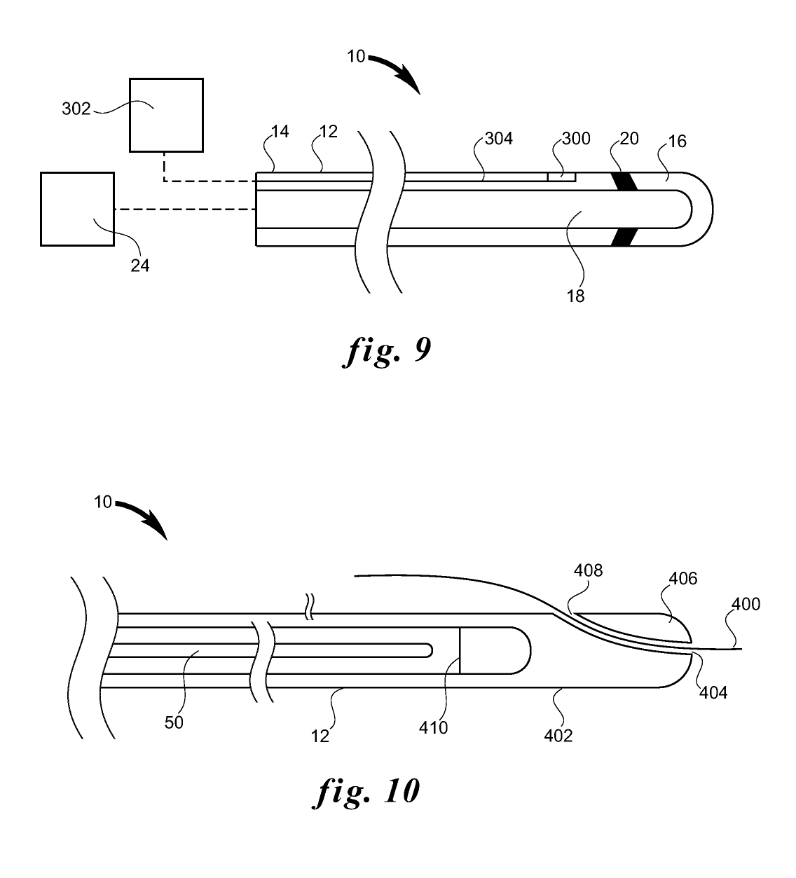

In any of the embodiments described herein, the device 10 may further include a pressure sensor 300 coupled to the elongated member 12 (FIG. 9). The device 10 is illustrated as having a similar configuration as that of FIG. 4, but in other embodiments, the device 10 may have other configurations, such as any of the other embodiments described herein. In the illustrated embodiments, the pressure sensor 300 is located at the distal end 16. In other embodiments, the pressure sensor 300 may be located at other locations. Also, in other embodiments, the device 10 may include a plurality of pressure sensors 300. For example, in some embodiments, the pressure sensors 300 may be all located at the distal end 16 (or at any of other locations along the length of the elongated member 12). In such cases, the pressure sensors 300 may be circumferentially disposed around the exterior surface of the elongated member 12. In other embodiments, the pressure sensors 300 may be disposed along the length of the elongated member 12. During use, the pressure sensor(s) 300 may be used to sense fluid pressure inside a vessel (e.g., intravascular pressure). A conductor 304 (e.g., a wire) may be used to transmit a signal representing the sensed pressure to a display device 302, such as a LCD or a flat panel, which displays the sensed pressure. The fluid pressure provides an indication whether there is clogging in the vessel, thereby allowing a physician to diagnose a medical condition.

In other embodiments, the pressure sensor(s) 300 may be used to determine fractional flow reserve (FFR), which is used in coronary catheterization to measure pressure differences across a coronary artery stenosis (narrowing, usually due to atherosclerosis) to determine the likelihood that the stenosis impedes oxygen delivery to the heart muscle. In some cases, the FFR is defined as the pressure behind (distal to) a stenosis relative to the pressure before the stenosis. The result is an absolute number. For example, a FFR of 0.50 means that a given stenosis causes a 50% drop in blood pressure. Thus, FFR expresses the maximal flow down a vessel in the presence of a stenosis compared to the maximal flow in the hypothetical absence of the stenosis.

In other embodiments, the pressure sensor(s) 300 may be used to determine coronary flow reserve (CFR), which is the maximum increase in blood flow through the coronary arteries above a normal resting volume. In some cases, its measurement by the sensor(s) 300 may be used to assist in the treatment of conditions affecting the coronary arteries and to determine the efficacy of treatment used.

In some embodiments, the same device 10 may be used to perform imaging of tissue within the vessel, to dilute blood inside the vessel during an imaging, or to do both. Also, in some embodiments, the same device 10 may be used to deliver a drug to treat the clogged vessel.

In other embodiments, the pressure sensor may be configured to sense a pressure for measuring Corinary Fractional Reserve (CFR) and/or Fractional Flow Reserve (FFR) in a vessel lumen.

In any of the embodiments described herein, the device 10 may be configured to be used with a guidewire. FIG. 10 illustrates the device 10 (which may be any of the embodiments of the device 10 described herein). The imaging window and the fluid delivery port(s) are not shown for clarity. However, it should be understood that the device 10 of FIG. 10 may include any of the embodiments of the fluid delivery port(s) described herein. Also, the shape of the device 10 may have any of the exemplary shapes described herein. The distal section 402 of the device 10 includes a port 404 at the distal tip 406, and a port 408 at a side of the device 10. During use, the guidewire 400 enters the port 404, and exits from the port 408 at the side of the device 10. The device 10 may optionally includes a partition/wall 410 for preventing fluid from the proximal end from entering the distal section 402 where the guidewire 400 is located. In other embodiments, the first port 404 and the second port 408 may be configured (e.g., arranged) such that the guidewire 400 may enter at the first port 404, and exit from the second port 408 in a linear configuration (FIG. 11). In one implementation, the device 10 may include a separate channel extending between the ports 404, 408, wherein the channel is located next to the imaging lumen in a side-by-side configuration.

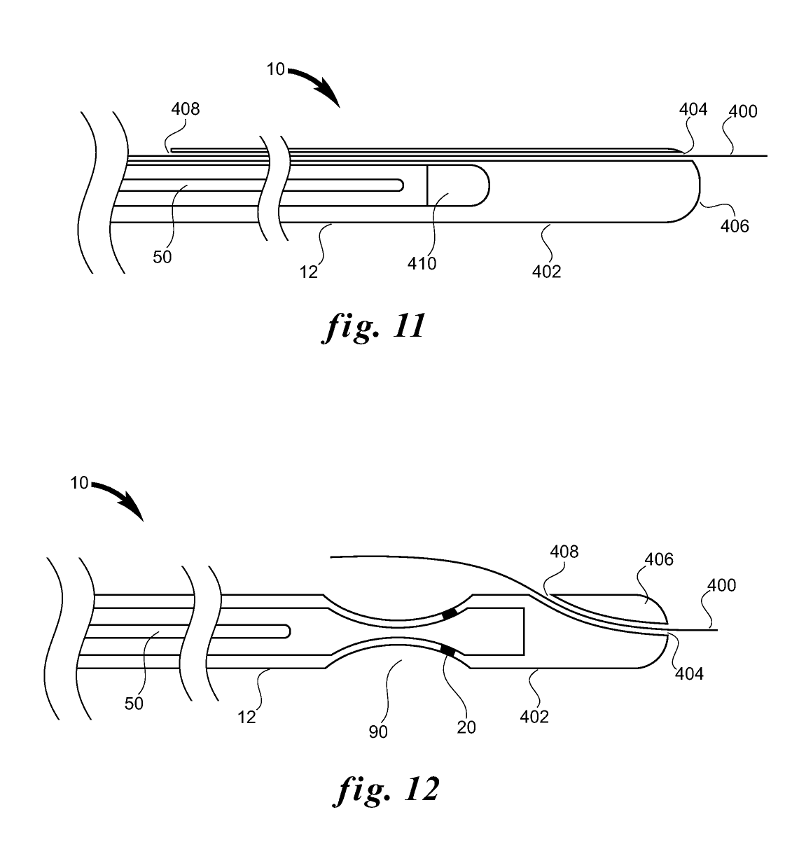

FIG. 12 illustrates another variation of the device 10, particularly showing the device 10 being used with a guidewire 400. The device 10 is similar to the device of FIG. 4A, except that the distal section 402 of the device 10 includes a port 404 at the distal tip 406, and a port 408 at a side of the device 10. During use, the guidewire 400 enters the port 404, and exits from the port 408 at the side of the device 10. The device 10 may optionally includes a partition 410 for preventing fluid from the proximal end from entering the distal section 402 where the guidewire 400 is located.

Although particular embodiments have been shown and described, it will be understood that they are not intended to limit the present inventions, and it will be obvious to those skilled in the art that various changes and modifications may be made without departing from the spirit and scope of the present inventions. The specification and drawings are, accordingly, to be regarded in an illustrative rather than restrictive sense. The present inventions are intended to cover alternatives, modifications, and equivalents, which may be included within the spirit and scope of the present inventions as defined by the claims.

What is claimed:

1. A device for delivering fluid inside a vessel, comprising:
an elongated member having a proximal end, a distal end, and a channel extending between the proximal end and the distal end; and
a first port located at the distal end, wherein the first port is in fluid communication with the channel;
wherein the first port faces at least partially towards a proximal direction.

2. The device of claim 1, wherein the channel is located within a wall of the elongated member.

3. The device of claim 1, wherein the elongated member comprises a tubular structure having a lumen, and the channel is located within the lumen.

4. The device of claim 1, further comprising a fluid source coupled to the proximal end of the elongated member, wherein the channel is configured to deliver fluid from the fluid source to the first port.

5. The device of claim 1, further comprising a second port located at the distal end, wherein the second port is in fluid communication with the channel.

6. The device of claim 1, wherein the elongated member comprises a wall at the distal end, and the port is at the wall of the elongated member.

7. The device of claim 6, wherein the wall faces towards a direction having a directional component that points towards the proximal direction.

8. The device of claim 6, wherein the distal end of the elongated member is without an enlarged profile.

9. The device of claim 1, further comprising an imaging device in an operative position relative to the elongated member.

10. The device of claim 9, wherein the elongated member is coupled to the imaging device.

11. The device of claim 9, wherein the imaging device is configured to perform an optical coherence tomography.

12. The device of claim 1, wherein the elongated member comprises an imaging window for collecting image signals.

13. The device of claim 12, wherein the first port is proximal to the imaging window, and is configured to deliver fluid from a first location that is proximal to the imaging window, to a second location that is proximal to the first port.

14. The device of claim 12, wherein the first port is distal to the imaging window, and is configured to deliver fluid from a first location that is distal to the imaging window, to a second location that is proximal to the first port.

15. The device of claim 12, further comprising an optical component in operative position relative to the imaging window.

16. The device of claim 15, wherein the optical component is moveable relative to the imaging window.

17. The device of claim 16, further comprising an additional imaging window, wherein the optical component is moveable from the imaging window to the additional imaging window.

18. The device of claim 15, wherein the optical component is a part of an optical coherence tomography device.

19. The device of claim 1, further comprising a pressure sensor coupled to the elongated member.

20. The device of claim 19, wherein the pressure sensor is configured to sense a pressure for determining a fractional flow reserve

21. The device of claim 19, wherein the pressure sensor is configured to sense a pressure for determining a coronary flow reserve.

22. A device for delivering fluid inside a vessel, comprising:
an elongated member having a proximal end, a distal end, and a channel extending between the proximal end and the distal end;
a first port located at the distal end, wherein the first port is in fluid communication with the channel; and
an imaging window;
wherein the first port is configured to deliver a fluid to dilute blood inside the vessel before the diluted blood reaches the imaging window.

23. The device of claim 22, wherein the channel is located within a wall of the elongated member.

24. The device of claim 22, wherein the elongated member comprises a tubular structure having a lumen, and the channel is located within the lumen.

25. The device of claim 22, further comprising a fluid source coupled to the proximal end of the elongated member, wherein the channel is configured to deliver fluid from the fluid source to the first port.

26. The device of claim 22, further comprising a second port located at the distal end, wherein the second port is in fluid communication with the channel.

27. The device of claim 22, wherein the elongated member comprises a wall at the distal end, and the port is at the wall of the elongated member.

28. The device of claim 27, wherein the wall faces towards a direction having a directional component that points towards the proximal direction.

29. The device of claim 27, wherein the distal end of the elongated member is without an enlarged profile.

30. The device of claim 22, further comprising an imaging device in an operative position relative to the elongated member.

31. The device of claim 30, wherein the elongated member is coupled to the imaging device.

32. The device of claim 30, wherein the imaging device is configured to perform an optical coherence tomography.

33. The device of claim 22, wherein the imaging window is at the elongated member.

34. The device of claim 33, wherein the first port is proximal to the imaging window, and is configured to deliver fluid from a first location that is proximal to the imaging window, to a second location that is proximal to the first port.

35. The device of claim 33, wherein the first port is distal to the imaging window, and is configured to deliver fluid from a first location that is distal to the imaging window, to a second location that is proximal to the first port.

36. The device of claim 33, further comprising an optical component in operative position relative to the imaging window.

37. The device of claim 36, wherein the optical component is moveable relative to the imaging window.

38. The device of claim 37, further comprising an additional imaging window, wherein the optical component is moveable from the imaging window to the additional imaging window.

39. The device of claim 36, wherein the optical component is a part of an optical coherence tomography device

40. The device of claim 22, further comprising a pressure sensor coupled to the elongated member.

41. The device of claim 40, wherein the pressure sensor is configured to sense a pressure for determining a fractional flow reserve

42. The device of claim 40, wherein the pressure sensor is configured to sense a pressure for determining a coronary flow reserve.
